(19) United States
(12) Patent Application Publication
(10) Pub. No.: US 2024/0416005 A1
(43) Pub. Date: Dec. 19, 2024

(54) MEDICAL DEVICE COMPRISING AN ASSEMBLY OF ACELLULAR BIOLOGICAL MATRIX ELEMENTS AND AT LEAST ONE POLYMER

(71) Applicant: PH TECH, LE TOURNE (FR)

(72) Inventors: Anthony PERES, DOMPIERRE SUR MER (FR); Guillaume HOFMANSKI, MIEUXCE (FR)

(21) Appl. No.: 18/020,725

(22) PCT Filed: Aug. 12, 2021
(86) PCT No.: PCT/EP2021/072473

§ 371 Date: Sep. 7, 2023

(30) Foreign Application Priority Data
Aug. 12, 2020 (FR) FR2008460

(51) Int. Cl. A61L 27/36; A61L 27/34

(52) U.S. Cl. CPC A61L 27/3604; A61L 27/34; A61L 2430/22

(57) ABSTRACT

The invention relates to a medical device comprising:
an assembly of at least two acellular biological matrix elements, and
at least one polymer.

The invention also relates to a method for manufacturing such a medical device and its use in particular as an implant, for example as a breast implant.

### TECHNICAL FIELD

The invention relates to the field of medical devices, and in particular three-dimensional implants with various shapes.

The subject of the invention is a particular medical device, its method of manufacture and its uses.

### STATE OF THE ART

Many surgical applications require soft tissue reinforcement and/or replacement of lost anatomical sections, such as breast reconstructions.

These procedures require the insertion of medical devices with elements combining mechanical resistance, flexibility and biological compatibility.

To this end, biological matrices are used more and more for the manufacture of medical devices on account of their biological compatibility.

For flat medical devices, the biological matrix can be used alone. However, for surgical indications requiring a particular three-dimensional shape, such as the placement of breast implants, the flat dermal matrix covers the implant/prosthesis.

Thus, currently, the use of an acellular biological matrix as a three-dimensional implant is only possible in the presence of a support (prosthesis, implant made of another material) allowing the biological matrix to be maintained in three dimensions. Moreover, current techniques are complex and require either perforating the acellular biological matrix to allow it to take on a three-dimensional shape, or cutting out elements of the matrix to assemble them by suturing, gluing, etc.

There is therefore a need for three-dimensional biological implants, of various shapes, in acellular biological matrix that do not require the presence of a mechanical support to maintain the biological matrix in three dimensions, combining mechanical integrity, optimal reinforcement, and adaptability for tissue reconstruction of complex shapes.

The objective of the invention is to meet this need by proposing a medical device in a dermal matrix overcoming the problems of the prior art.

### SUMMARY OF THE INVENTION

To this end, the subject of the invention is a medical device comprising:
an assembly of at least two acellular biological matrix elements, and
at least one polymer.

In particular, the acellular biological elements are assembled and maintained by at least one layer comprising at least one polymer: one or more layer(s) of polymer(s) partially or totally around the assembly of the two acellular biological matrix elements and/or one or more layer(s) of polymer(s) partially or totally between the two acellular biological matrix elements.

The invention thus relates to a device obtained by assembling various elements cut out from acellular biological matrices, and maintained by at least one polymer.

Advantageously, in addition to allowing the maintenance of different acellular biological matrix elements, the presence of at least one polymer makes it possible to render the biological matrix resistant to infections while retaining the qualities of the biological matrix in terms of mechanical resistance, flexibility, and biological compatibility. The medical device according to the invention is thus particularly suitable and advantageous for medical applications, in particular in surgery.

According to one aspect, the invention also relates to a method for manufacturing such a medical device. The method comprises at least the implementation of the following steps:
a. taking at least one acellular biological matrix and optionally preparing it on at least part of the matrix so as to allow the adhesion of a solution comprising at least one polymer on said part,
b. cutting out at least two acellular biological matrix elements in the acellular biological matrix or matrices of step a.
c. if the cellular biological matrix part(s) in which the acellular biological matrix elements that were cut out in step b. have not been prepared in step a., then at least one acellular biological matrix element is prepared at least on one part so as to allow the adhesion of a solution comprising at least one polymer on said part,
d. optionally coating at least one acellular biological matrix element in whole or in part with a solution comprising at least one polymer,
e. assembling the acellular biological matrix elements together in a given form,
f. necessarily if step d. has not been performed, or optionally if step d. has been performed: partially or totally coating at least one acellular biological matrix element and/or the assembly of acellular biological matrix elements with a solution comprising at least one polymer.

Other features and advantages will emerge from the detailed description of the invention that follows.

### BRIEF DESCRIPTION OF THE FIGURES

FIG. 1 shows an example of a medical device according to the invention, consisting of acellular biological matrix layers of different sizes, superimposed on each other, held together by polymer layers, to form a breast implant.

### DETAILED DESCRIPTION OF THE INVENTION

### Definitions

Within the meaning of the invention, the term “acellular” biological matrix is intended to refer to a biological matrix in which the cellular elements have been eliminated through a decellularization process with the aim of destroying and/or removing the cells and their components from the extracellular matrix of the biological matrix while maintaining its structure and properties. In fact, in order for a biological matrix to be implanted in a recipient, it must be decellularized so as to decrease its immunogenicity.

Within the meaning of the invention, the term “allograft” is understood to refer to a biological matrix—a graft—originating from a donor belonging to the same biological species as the recipient.

The term “acellular biological matrix element” is understood to refer to all or part of an acellular biological matrix. Preferably, it is an element cut out or formed in an acellular biological matrix.

Within the meaning of the invention, the term “implant” is understood to refer to a medical device used in surgery.

Within the meaning of the invention, the term “strip” is understood to mean a layer of acellular biological matrix of variable thickness, solid or hollowed out, of a certain geometric shape.

Within the meaning of the invention, the term “biological matrix” is understood to refer to a biomaterial derived from the human or animal species.

Within the meaning of the invention, the term “P4HB” is understood to refer to poly-4-hydroxybutyrate, a specific PHA. It is a homopolymer of a 4-hydroxybutyrate unit.

The term “peel test” is used here to refer to a test making it possible to determine the strength of adhesion between two materials. Each material is placed in pneumatic jaws at a given pressure and then separated at constant speed as specified in the examples.

Within the meaning of the invention, the term “PHA” is understood to refer to polyhydroxyalkanoates, which are biodegradable polyesters.

Within the meaning of the invention, the term “solution” is understood to refer to a homogeneous mixture resulting from the dissolution of one or more solutes in a solvent.

Within the meaning of the invention, the term “suture retention force” or “suture retention strength” is understood to refer to a test for determining the force (N) required to pull a suture out of a specimen.

Within the meaning of the invention, the term “uniaxial tensile strength” is understood to refer to a test for determining the tension prior to the rupturing of the specimen being tested. The properties measured are ultimate tensile strength, breaking strength, and elongation at rupture.

Within the meaning of the invention, the term “viscosity” is understood to refer to a property of resistance to the flow of a fluid for non-turbulent flow.

Within the meaning of the invention, the term “xenograft” is understood to refer to a biological matrix—a graft—originating from a donor belonging to a biological species different from that of the recipient.

### Medical Device According to the Invention

According to a first aspect, the invention therefore relates to a medical device comprising:
an assembly of at least two acellular biological matrix elements, and
at least one polymer.

Acellular biological matrices make up a large class of biomaterials that are extracted from grafts of various origins.

Preferably, the biological matrix or matrices of the biological matrix elements in the medical device according to the invention are of human and/or non-human animal origin (allograft or xenograft).

According to a particularly suitable embodiment, the biological matrix or matrices of the biological matrix elements are selected from among biological matrices of non-human animal origin, preferably selected from among biological matrices of porcine, bovine, equine, caprine, or fish origin, and mixtures thereof.

Preferably, the biological matrix or matrices of the biological matrix elements are selected from among all animal and/or human biological matrices, preferably from one of the following biological matrices: dermis, intestinal submucosa, aorta, bladder, amniotic membrane, peritoneum, pericardium, dura mater, tendons, bones, cartilage, and mixtures thereof.

The biological matrix or matrices of the biological matrix elements of the medical device according to the invention are acellular. There are many known methods for obtaining an acellular biological matrix. The methods used may be enzymatic and/or based on chemical solutions and/or on mechanical processes. The method used must be a method that makes it possible to obtain an acellular biological matrix that is capable of being used in surgery, particularly for the reconstruction of soft tissues.

The biological matrix or matrices of the biological matrix elements of the medical device according to the invention are preferably chosen from acellular biological matrices that have at least one of the following characteristics, even more preferably all of them:
uniaxial tensile strength greater than or equal to 5 N/mm² for a 5 mm×50 mm specimen with a thickness taken into account in the calculation. Each end is secured in pneumatic grips lengthwise 1 cm from each edge. The edge spreading rate is 30 mm/min. Uniaxial tensile strength is found by dividing the maximum force (N)/(5 (mm)×thickness (mm)).
suture retention force greater than or equal to 5 N for a 1 cm×1 cm sample using an appropriate suture (4-0 polypropylene-type) threaded 3 mm from the edge of the sample in its center, the opposite side being clamped in a pneumatic grip at approximately 5 mm. The two ends of the suture are clamped in the lower grip. The grip separation speed is 20 mm/min. The maximum force (N) is noted.

Preferably, the biological matrix or matrices used has or have the specifications defined in the applicable standards (“USP official monographs” latest version, ASTM) according to the type of biological matrix used.

Preferably, the acellular biological matrix elements of the device according to the invention:
are dry, preferably with a residual moisture content of between 10% and 18%, and/or
they have, at least on one part, a surface appearance that improves the adhesion of at least one polymer to the acellular biological matrix, in particular the adhesion of at least one layer of polymer solution.

According to a variant of the invention, all the acellular biological matrix elements come from an identical biological matrix. According to another variant of the invention, at least two acellular biological matrix elements come from different biological matrices.

The acellular biological matrix elements of the device according to the invention may have different shapes (round, square, circular, irregular, etc.), be flat or in relief, possibly with channels or holes or any hollow geometric shape (inclusion), hollowed out completely or partially inside, or may have at least one textured surface. Preferably, each acellular biological matrix element has a varied and variable shape that may be different or identical in the same medical device. Their shapes may be obtained by cutting in an acellular biological matrix. The acellular biological matrix elements may be produced according to the plans of a 3D file of the final medical device specifying the shape and dimensions of each matrix element to be assembled.

Each acellular biological matrix element is preferably in the form of a layer. Thus, the acellular biological matrix elements are layers of acellular biological matrices. Preferably, the acellular biological matrix elements are strips of acellular biological matrices.

Each acellular biological matrix element has a variable size and a regular or irregular thickness, the size and the thickness possibly being different or identical in the same medical device. Preferably, the acellular biological matrix elements have a maximum thickness of between 0.5 and 5 mm. According to a variant, at least one acellular biological matrix element is an acellular biological matrix layer or strip, of variable thickness, the maximum thickness of which is between 0.5 and 5 mm. According to another variant, at least one acellular biological matrix element is an acellular biological matrix layer or strip, of constant thickness, the constant thickness of which is between 0.5 and 5 mm.

In the medical device according to the invention, at least two acellular biological matrix elements are assembled together to form an assembly.

Preferably, the acellular biological matrix elements are superimposed on each other horizontally or are aligned vertically next to each other. When two acellular biological matrix elements are superimposed on each other, it is all or part of one of the acellular biological matrix elements that is superimposed on all or part of the other acellular biological matrix element. When two acellular biological matrix elements are aligned next to each other, it is all or part of one of the acellular biological matrix elements that is aligned vertically next to all or part of the other acellular biological matrix element.

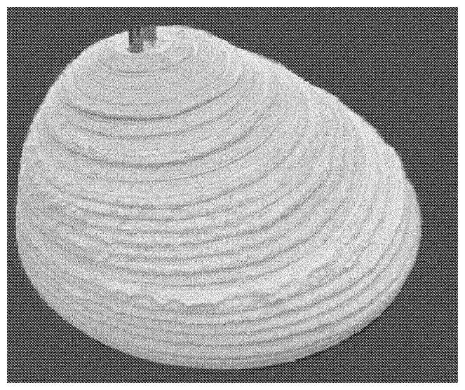

An example of a medical device with biological matrix strips superimposed on each other is shown in the photo of FIG. 1. The strips are circles of different sizes and identical thicknesses, placed on top of each other, from largest to smallest, to form a breast implant.

The cellular biological matrix elements in the device according to the invention are held together, assembled owing to the presence of at least one polymer, preferably at least one polymer layer. Thus, the polymer may be placed in the medical device in one or more layers.

Preferably, the polymer:
partially or totally covers at least one acellular biological matrix element (it may in particular be present partially or totally between at least two acellular biological matrix elements), and/or
partially or totally covers each acellular biological matrix element
partially or totally covers the assembly of acellular biological matrix elements.

According to a variant, the device according to the invention is preferably in a laminated-glued form: acellular biological matrix strips maintained, assembled by partial or total coating with a polymer (coating of the final outer envelope and/or depositing layers of polymers between one or more of the matrix strips.

The polymer present in the medical device according to the invention can be any type of polymer that is suitable for use as a medical device and particularly as a surgical implant.

The polymer or polymers present in the device according to the invention are preferably selected from among the following polymers: poly(glycolides), poly(lactide-co-glycolides); poly(lactic acid), poly(glycolic acid), poly(lactic acid-co-glycolic acids), polycaprolactones, poly(orthoesters), polyanhydrides, poly(phosphazenes), polyhydroxyalkanoates (including, in particular, P4HB and poly-3-hydroxybutyrate-co-3-hydroxy valerate (PHBV)), polyesters, poly(lactide-co-caprolactones), polycarbonates, tyrosine polycarbonates, polyamides, polyesteramides, poly(dioxanones), poly(alkylene alkylates), polyethers, polyvinyl pyrrolidones or PVP, polyurethanes, polyether esters, polyacetals, polycyanoacrylates, poly(oxyethylene)/poly(oxypropylene) copolymers, polyacetals, polyketals, polyphosphates, polyphosphoesters, polyalkylene oxalates, polyalkylene succinates, poly(maleic acids), chitin, chitosan, and mixtures thereof.

According to a particularly suitable embodiment, the device according to the invention comprises at least one PHA, and even more preferably a PHA that is selected from among at least P4HB, the copolymers of P4HB, and mixtures thereof. PHAs are a family of materials produced by many microorganisms. One noteworthy example is U.S. Pat. No. 6,316,262 of Metabolix, Inc. from Cambridge, MA, USA, which describes a method for obtaining a biological system that enables the production of polyhydroxyalkanoate polymers containing 4-hydroxy acids. U.S. Pat. Nos. 6,245,537, 6,623,748, 7,244,442 and 8,231,889 also describe methods for producing PHAs. Preferably, PHA and in particular P4HB and/or its copolymers have a low level of endotoxins (less than 20 EU/device).

The device according to the invention may therefore comprise one or more acellular biological matrices, one or more polymer layer(s) and possibly other constituents, each acellular biological matrix element possibly being completely or partially covered by one or more polymer layer(s).

The presence of the polymer(s) in the medical device enables optimal reinforcement to be achieved during tissue reconstruction supported by the biological matrix, even in the event of infection.

The polymer layer(s) may comprise one or more channels, cavities or hollow elements of variable geometry that make it possible, during use in surgery, to incorporate elements promoting optimal construction of the tissues in which the device is used as an implant (PRP, stem cells, antibiotics, adipose tissue, etc.). They may be circular or of different geometries. They may be obtained by imprinting a form in the biological matrix through pressing before coating of the biological matrix. The form is then removed after coating and drying.

The medical device in its final form (breast implant, tube for trachea replacements, etc.) may also include channels, cavities, or any hollow element of variable geometry inside. These elements (channels, cavities, or any element of variable geometry inside) may be integrated by cutting into each of the acellular biological matrix elements to obtain the desired final geometry of the medical device.

The channels, cavities, or any hollow element of variable geometry inside the medical device and/or the polymer layer(s) and/or the acellular biological matrix layer(s) preferentially have an internal surface between 0.007 mm² and 1 mm².

### Method for Manufacturing a Medical Device According to the Invention

The medical device according to the invention may be obtained by all suitable processes. In particular, the invention relates to a method for manufacturing a medical device comprising implementation of the following steps:
a. taking at least one acellular biological matrix and optionally preparing it on at least part of the matrix so as to allow the adhesion of a solution comprising at least one polymer on said part,
b. cutting out at least two acellular biological matrix elements in the acellular biological matrix or matrices of step a, optionally including cutouts for creating channels, cavities, or any hollow element of variable geometry (inclusions with variable geometries) inside the medical device (after assembly step e),
c. if the cellular biological matrix part(s) in which the acellular biological matrix elements were cut out have not been prepared in step a., then at least one acellular biological matrix element is prepared at least on one part so as to allow the adhesion of a solution comprising at least one polymer on said part,
d. optionally coating at least one acellular biological matrix element in whole or in part with a solution comprising at least one polymer,
e. assembling the acellular biological matrix elements together in a given form,
f. necessarily if step d. has not been performed, or optionally if step d. has been performed: partially or totally coating at least one acellular biological matrix element and/or the assembly of acellular biological matrix elements with a solution comprising at least one polymer.

The acellular biological matrix or matrices at the start of the method, in step a., may have different shapes (round, square, circular, irregular, etc.), be flat or raised, and/or with channels, cavities, or hollow elements of variable geometries (inclusions with variable geometries) and/or with one surface textured, and be of regular or irregular thickness.

Preferably, at least one of the acellular biological matrices of step a. is a planar acellular biological matrix. According to one embodiment, all the acellular biological matrices of step a., if there are several, are planar.

In a preferred embodiment, the acellular biological matrix, prior to coating and/or after coating, should be dry or dried in such a way as to have a residual moisture content in the range of 10% to 18%. The residual moisture content is preferably measured using a Mettler Toledo brand halogen moisture analyzer-type desiccator.

One drying technique used is preferably that of loss on drying described in USP 41 (bovine dermis scaffold):
1—an empty aluminum dish is positioned in the device and taring is carried out,
2—the dish is filled with a sample of 1.0 g+/−0.2 g cut into 4 mm² pieces,
3—the heating program at 130° C. is launched
4—when the weight no longer varies over a given time, the result is displayed in %.

Another conventional oven-drying method may also be used. In this context, an aluminum dish previously weighed empty is filled with a sample of 5.0 g+/−0.2 g cut into 4 mm² pieces. The entire thing is heated at 100° C. for 16 h. It is then weighed and the loss on drying is calculated:

dry matter %=[(weight of the dry extract+dish (g)−weight of dish (g))/g of sample]×100

moisture %=100−dry matter %.

The acellular biological matrix or matrices and/or the acellular biological matrix elements, in whole or in part, before coating, must be prepared so as to allow the adhesion of one or more polymer solution(s).

Preferably, the preparation of the cellular biological matrix in step a. or in step c. consists of a chemical and/or mechanical and/or electrochemical and/or physical surface treatment. For example, it may be a treatment by abrasion and/or milling and/or microtexturing and/or laser and/or UV.

The prepared biological matrix or matrices or biological matrix elements may then be used as a support for coating one or more polymer solution(s) in step d. and/or in step f.

The polymer or polymers used for coating the acellular biological matrix or matrices or the acellular biological matrix element(s) in the method according to the invention are preferably selected from among the following polymers: poly(glycolides), poly(lactide-coglycolides); poly(lactic acid), poly(glycolic acid), poly(lactic acid-co-glycolic acids), polycaprolactones, poly(orthoesters), polyanhydrides, poly(phosphazenes), polyhydroxyalkanoates (including, in particular, P4HB and poly-3-hydroxybutyrate-co-3-hydroxy valerate (PHBV)), polyesters, poly(lactide-co-caprolactones), polycarbonates, tyrosine polycarbonates, polyamides, polyesteramides, poly(dioxanones), poly(alkylene alkylates), polyethers, polyvinyl pyrrolidones or PVP, polyurethanes, polyether esters, polyacetals, polycyanoacrylates, poly(oxyethylene)/poly(oxypropylene) copolymers, polyacetals, polyketals, polyphosphates, polyphosphoesters, polyalkylene oxalates, polyalkylene succinates, poly(maleic acids), chitin, chitosan, and mixtures thereof.

Preferably, the solution comprising at least one polymer was obtained previously through solubilization of the dry polymer(s) in at least one solvent, preferably at least one polar solvent.

In fact, the polymer(s) must be preferably converted into a solution that may enable coating using the appropriate solvent.

When the polymer is a PHA and, in particular, P4HB and/or one of its copolymers, the solvent is preferably selected from among the following polar solvents: dichloromethane, chloroform, tetrahydrofuran, dioxane, acetone, and mixtures thereof.

In a preferred embodiment, the P4HB is dissolved in an acetone solution, preferably in a ratio of from 5% to 25% (w/w). The respective amounts are combined, heated, and maintained at a temperature below the boiling point of acetone (approximately 56° C.) until the P4HB dissolves completely and the desired viscosity is obtained, at least 10%, more preferably 15%, and even more preferably 20% (w/w).

Preferably, after dissolving the polymer(s) in a solvent, the polymer solution is degassed and/or debubbled in order to purge the mixture of air bubbles. Preferably, the solution is placed under vacuum (minimum −1 bar) for the time required for complete degassing/debubbling.

The step of coating the acellular biological matrix with a polymer solution is preferably carried out at a temperature less than or equal to the denaturation temperature of the collagen. In a particularly suitable manner, the coating is carried out at a temperature between 1° and 60° C., preferably between 1° and 50° C., even more preferably between 2° and 50° C.

The coating may be performed by any suitable means, preferably by solvent casting, spray coating, or dip coating.

Advantageously, the method according to the invention allows for the direct application of a polymer solution at the desired concentration to an acellular biological matrix or a previously prepared acellular biological matrix element, and it is not necessary to make a polymer film or sheet beforehand and then place it on the support in order to allow adhesion of the elements to each other by heating.

When it is carried out by solvent casting, the coating may be carried out using different technologies: knife, double side, commabar, case knife, engraved roller, 2 rollers, 3 roller combi, microroller, 5 rollers, reverse roller, rotary screen, dipping, slot die, curtain coating, or hot-melt slot die. The simplest method is based on the use of an Elcometer-type casting knife. The biological matrix is placed on a table. The quantity of polymer solution required as a function of the desired thickness and the surface to be treated is placed on the acellular biological matrix. The gardener knife is then moved over the acellular biological matrix in order to make the coating on the implant uniform. The gardener knife has been adjusted beforehand to a certain height.

In order to automate the operation, a coating machine may be used. The polymer solution is pumped through a slot die in order to be applied to the moving biological implant. In a preferred embodiment, the width of the slot die is 600 mm, and the conveyance speed of the implant is from 1-10 m/min. The pumping speed, the conveyance speed, the width of the slot die, and the concentration of the solution may be adjusted in order to obtain an implant with a coating of the desired thickness and width.

When the coating is carried out by spray coating or atomization, the polymer solution is pumped up to a nozzle that projects droplets onto the surface to be treated. This technique is particularly advantageous for the treatment of biological matrices having 3D shapes (example: biological implant having a hemispherical, ovoid, tubular shape, or in the form of anatomical breast implants, etc.).

When the coating is performed by means of dip coating, or immersion, the piece to be treated is dipped in a dissolved, melted, softened, or fluidized powder material in order to cover it with a layer of that material. This technique is particularly advantageous for the treatment of raised and planar biological implants.

After step d, the device according to the invention may already have its final shape, the latter being dictated by the shape and dimensions of the acellular biological matrix elements and by the assembly of said elements.

According to a particular embodiment of the invention, a 3D file of the desired shape is produced. The shape is solid or made up of channels, cavities, or other hollow geometric inclusions (hollow element of variable geometry). The shape is then decomposed into different constituent acellular biological matrix elements. This 3D file then allows the corresponding acellular biological matrix elements to be cut out, which are to be assembled to form the medical device to the desired final shape. The elements are therefore then assembled according to the 3D file. According to a specific example of a variant, as shown in FIG. 1 with the example of a medical device for use as a breast implant, the acellular biological matrix elements constituting the medical device are held in position by the use of a guide passing through all of said elements.

According to another particular embodiment of the invention, the acellular biological matrix elements are not cut out so as to form the medical device according to the invention once assembled. In this embodiment, the acellular biological matrix elements are assembled (superposed or juxtaposed) preferably to form a block and the method comprises, after step e. (before or after step f.), a step of cutting out, trimming or sculpting, in the object formed by the assembly of acellular biological matrix elements and optionally of polymer(s), to obtain a different shape corresponding to the desired shape for the medical device according to the invention.

Optionally, after step f., the medical device obtained is then:
dried, on the one hand, and/or
pressed to obtain a uniform thickness, on the other hand.

For drying, the medical device according to the invention may be placed in an oven or a furnace or a heating chamber so as to allow the complete evaporation of the solvent, at a temperature of between 0° C. and 50° C., between 15 and 40° C., preferably 30° C. plus or minus 5°, so as to prevent excessively rapid evaporation and deformation of the biological matrix.

For pressing, the medical device according to the invention is pressed in a hydraulic press over a period of 30 to 60 s, between 50 bars and 200 bars and at a temperature between 50° C. and 100° C. According to a variant, it is not the medical device that is pressed, but the acellular biological matrices at the start of the method or the cellular matrix elements at any time during the method. When the matrices or matrix elements are flat, they can be pressed in a hydraulic press between two plates. The biological matrices in relief or the biological matrix elements in relief (non-flat) or the medical devices according to the invention can be pressed in molds having the desired imprints (for example, of the molding machine for fabric cup type).

### Use of the Medical Device According to the Invention

The medical device according to the invention can be used for any medical application, in particular as an implant, particularly in surgery. It may be used as such or as an implant, in particular as a surgical biological implant. According to a variant, it is for example an implant for breast reconstruction or an implant in the form of a tube for tracheal replacements.

In particular and without limitation, the medical devices according to the invention can be used for the following applications: repair, regeneration, and replacement of soft and hard tissues, healing device, bandage, patch, dressing, dressing for burns, dressing for ulcers, skin substitute, hemostatic, tracheal reconstruction device, organ rescue device, dural substitute, dural patch, nerve guide, nerve regeneration or repair device, hernia repair device, hernia mesh, hernia plug, temporary wound or tissue support device, tissue engineering scaffolding, guided tissue repair/regeneration device, mesh fixation devices, non-stick membrane, adhesion barrier, tissue separation membrane, retention membrane, sling, pelvic floor reconstruction device, urethral suspension device, urinary incontinence treatment device, bladder repair device, bulking or filling device, rotator cuff repair device, meniscus repair device, meniscus regeneration device, guided tissue regeneration membrane for periodontal tissue, anastomosis device, cell-seeded device, cell encapsulation device, controlled release device, drug delivery device, plastic surgery device, breast lift device, mastopexy device, breast reconstruction device, breast augmentation device, breast reduction device, breast reconstruction devices after mastectomy with or without breast implants, rhinoplasty device.

The invention will now be illustrated by examples.

### EXAMPLES

### Example 1: Treatment of an Acellular Biological Matrix

A dry, planar, acellular dermal matrix (residual moisture content between 10% and 18%) is placed on a numerically controlled machine tool. A carbide cutter measuring 5 mm in diameter is mounted on the machine tool. A rotation speed of from 20,000 to 40,000 rpm is used with an advancement of 2 m/min. The depth is variable depending on the desired final thickness. The surfacing may be total or concern only a portion of the implant so as to define forms.

### Example 2: Treatment of an Acellular Biological Matrix

A dry, planar, acellular dermal matrix (residual moisture content between 10% and 18%) is placed on a brushing/carding machine. The surface of the implant is thus treated with cards of desired diameter (105 mm, for example) composed of metallic wires (having a diameter of 0.2 mm, for example).

### Example 3: Treatment of an Acellular Biological Matrix

A planar acellular biological matrix is placed in a digital cutting machine with an oscillating knife. An oscillating knife cuts each strip according to the 3D file produced, including or not including geometric elements.

### Example 4: Treatment of an Acellular Biological Matrix

A dry, acellular dermal matrix (residual moisture content between 10% and 18%) is treated with a Telea Biotech-type high-frequency electrical field (4-64 MHZ) so as to create cavities and/or perforations 0.6 mm in diameter.

### Example 5: Formation of Acellular Biological Matrix Elements and Coating

A strip is cut from an acellular dermal matrix treated according to example 1 in a circle 12 cm in diameter. This strip is placed on a table. Since the desired amount of P4HB is 0.0164 g/cm², an 18% P4HB/acetone solution (w/w) is prepared. The required amount is removed and deposited on the implant. The casting knife is then moved translationally on the implant in order to make the thickness of the coating uniform. A coating machine may be used to automate the operation. An acetone/P4HB solution is prepared in order to supply the machine. The solution is pumped through a slot die in order to be applied to the moving planar biological implant. In a preferred embodiment, the width of the slot die is 600 mm (adapted to the width of the part to be treated); the conveyance speed of the implant is 1-10 m/min. The next strip is then added to the polymer still in the viscous state in order to allow adhesion. Pressure may be exerted to promote adhesion. The coating operation may be repeated on the top layer to add an additional layer.

### Example 6: Example of a Dip Coating Process

A breast implant consisting of 24 strips of acellular biological matrices obtained for example according to example 5 is held assembled by a crosspiece. The crosspiece is fixed on a dip coating machine in a vertical position and then immersed by a machine in a tank containing an acetone/P4HB solution at the desired concentration. The breast implant is then extracted from the tank by an upward vertical movement at a speed of 25 mm/min. The density of P4HB obtained by coating is then from 0.02 to 1 g/cm².

### Example 7: Example of a Dip Coating Process

A breast implant (like the one of FIG. 1) consisting of 24 strips of acellular biological matrices obtained for example according to example 5 is held assembled by a crosspiece. The implant is next immersed in a tank containing an acetone/P4HB solution at the desired concentration. The breast implant is then extracted from the tank and subjected to planetary rotation so as to standardize the polymer layer.

### Example 8: Example of Drying and Pressing and Characteristics of the Medical Devices Obtained

A strip of dermal matrix obtained according to example 5 is coated with approximately 0.03 g/cm² of P4HB and is pressed for 30 s at 100 bar of pressure and at 50° C. (previously heated press plates). On three test pieces tested, with an average thickness of 1.35 mm, the maximum average uniaxial tensile strength (UTS) is 26.50 MPa.

The test is carried out on an Instron measurement bench model 3342/L2345. The specimen is cut with a bone-shaped type V cutter as described in standard ASTM D-638-5. The piece cut in this manner is inserted by each end into the pneumatic grips of the bench (60 psi), leaving a central portion of 2.5 cm. A speed of 25 mm/min is applied until the piece breaks. The uniaxial tensile strength is noted (max. force/sectional area).

A T-peel test is carried out. Specimens of 2 cm×6 cm are cut out. The P4HB coating is separated from the biological implant on a section measuring 1.5 cm×2 cm. The two pieces separated in this manner are placed in the pneumatic grips (45 psi). The section of the coated biological implant is separated at a speed of 25 mm/min. The T-peel force is measured over a standard width of 20 mm and on an average of 5 peaks (loads). The adhesion was stronger over the three specimens that were tested; the coated layer did not manage to be peeled in the test.

### Example 9: Example of Drying and Pressing and Characteristics of the Medical Devices Obtained

Employing the same conditions as in the preceding tests, a strip cut out in a dermal matrix that has been treated according to example 2 is coated with approximately 0.03 g/cm² of P4HB and then pressed for 60 s at 200 bar and 100° C., resulting in an average UTS max value over three specimens of 27.65 MPa.

### Example 10: Example of Drying and Pressing and Characteristics of the Medical Devices Obtained

Employing the same conditions as in the preceding tests, a strip cut out in a dermal matrix that has been treated according to example 3 is coated with approximately 0.03 g/cm² of P4HB and then pressed for 30 s at 100 bar and 100° C., resulting in an average UTS max value over three specimens of 33.07 MPa.

1. A medical device comprising:
an assembly of at least two acellular biological matrix elements, and
at least one polymer.

2. The medical device as set forth in claim 1, characterized in that the polymer partially or totally covers at least one acellular biological matrix element.

3. The medical device as set forth in claim 1, characterized in that the polymer partially or totally covers the assembly of acellular biological matrix elements.

4. The medical device as set forth in claim 1, characterized in that the polymer is placed in one or more layers.

5. The medical device as set forth in claim 1, characterized in that the acellular biological matrix elements are layers of acellular biological matrix.

6. The medical device as set forth in claim 1, characterized in that the acellular biological matrix elements are strips of acellular biological matrices.

7. The medical device as set forth in claim 1, characterized in that the acellular biological matrix elements have a maximum thickness of between 0.5 and 5 mm.

8. The medical device as set forth in claim 1, characterized in that the acellular biological matrix elements are superimposed on each other horizontally or are aligned vertically one beside the other.

9. The medical device as set forth in claim 1, characterized in that all the acellular biological matrix elements come from an identical biological matrix.

10. The medical device as set forth in claim 1, characterized in that at least two acellular biological matrix elements come from different biological matrices.

11. The medical device as set forth in claim 1, characterized in that the acellular biological matrix elements are biological matrix elements of human and/or animal origin.

12. The medical device as set forth in claim 11, characterized in that at least one acellular biological matrix element is an acellular biological matrix element of animal origin selected from among the acellular biological matrices of porcine, bovine, equine, caprine, or fish origin, and mixtures thereof.

13. The medical device as set forth in claim 1, characterized in that at least one acellular biological matrix element is an acellular biological matrix element selected from the following biological matrices: dermis, intestinal submucosa, aorta, bladder, amniotic membrane, peritoneum, pericardium, dura mater, tendons, bones, cartilage, and mixtures thereof.

14. The medical device as set forth in claim 1, characterized in that the polymer(s) are selected from among the following polymers: polyhydroxyalkanoates, poly(glycolides), poly(lactide-co-glycolides), poly(lactic acid), poly(glycolic acid), poly(lactic acid-co-glycolic acids), polycaprolactones, poly(orthoesters), polyanhydrides, poly(phosphazenes), polyhydroxyalkanoates, polyesters, poly(lactide-co-caprolactones), polycarbonates; tyrosine polycarbonates, polyamides, polyesteramides, poly(dioxanones), poly(alkylene alkylates), polyethers, polyvinyl pyrrolidones, polyurethanes, polyether esters, polyacetals, polycyanoacrylates, poly(oxyethylene), copolymers of poly(oxypropylene), polyacetals, polyketals, polyphosphates, polyalkylene oxalates, polyalkylene succinates, poly(maleic acids), chitin, chitosan, and mixtures thereof.

15. The medical device as set forth in claim 1, characterized in that the polymer(s) are selected from among the following polymers: P4HB, co-polymers, and mixtures thereof.

16. The medical device as set forth in claim 1, characterized in that at least one acellular biological matrix element comprises one or more channels or cavities or hollow elements of variable geometry.

17. The medical device as set forth in claim 1, characterized in that it comprises one or more channels, cavities, or hollow or solid elements of variable geometry.

18. The medical device as set forth in claim 16, characterized in that the channels, cavities, or hollow or solid element of variable geometry have an internal surface of between 0.007 mm² and 1 mm².

19. A method for manufacturing a medical device as set forth in claim 1, characterized in that it comprises carrying out the following steps:
a. taking at least one acellular biological matrix and optionally preparing it on at least part of the matrix so as to allow the adhesion of a solution comprising at least one polymer on said part,
b. cutting out at least two acellular biological matrix elements in the acellular biological matrix or matrices of step a., optionally including cutouts intended to create channels, cavities, or any hollow element of variable geometry inside the medical device,
c. if the cellular biological matrix part(s) in which the acellular biological matrix elements were cut out have not been prepared in step a., then at least one acellular biological matrix element is prepared at least on one part so as to allow the adhesion of a solution comprising at least one polymer on said part,
d. optionally coating at least one acellular biological matrix element in whole or in part with a solution comprising at least one polymer,
e. assembling the acellular biological matrix elements together in a given form,
f. necessarily if step d. has not been performed, or optionally if step d. has been performed: partially or totally coating at least one acellular biological matrix element and/or the assembly of acellular biological matrix elements with a solution comprising at least one polymer.

20. The method for manufacturing a medical device as set forth in claim 19, characterized in that after step e., it comprises a cutting step in the object formed by assembling acellular biological matrix elements and polymer so as to obtain a different shape.

21. The method for manufacturing a medical device as set forth in claim 19, characterized in that at least one of the acellular biological matrices of step a. is a planar acellular biological matrix.

22. The method for manufacturing a medical device as set forth in claim 19, characterized in that the coating is performed at a temperature lower than or equal to the denaturation temperature of the collagen.

23. The method for manufacturing a medical device as set forth in claim 22, characterized in that the coating is performed at a temperature between 1° and 60° C.

24. The method for manufacturing a medical device as set forth in claim 19, characterized in that the biological matrix is dried so as to have a residual moisture content of between 10% and 18%.

25. The method for manufacturing a medical device as set forth in claim 19, characterized in that the step of preparing the cellular biological matrix consists of a chemical and/or mechanical and/or mechanical and/or electrochemical and/or physical surface treatment.

26. The method for manufacturing a medical device as set forth in claim 19, characterized in that the coating is carried out by solvent casting, spray coating, or dip coating.

27. The method for manufacturing a medical device as set forth in claim 19, characterized in that the solution comprising at least one polymer was obtained previously through solubilization of the polymer(s) in at least one polar solvent.

28. The method for manufacturing a medical device as set forth in claim 27, characterized in that, after solubilization of the polymer(s), the solution is degassed and/or debubbled under vacuum in order to purge the mixture of air bubbles.

29. The method for manufacturing a medical device as set forth in claim 19, characterized in that the polymer solution comprises at least P4HB dissolved in an acetone solution.

30. An implant comprising the medical device of claim 1.

31. The implant of claim 30, wherein the implant is a breast implant or implant in the form of a tube for trachea replacement.
